(19) United States
(12) Patent Application Publication
(10) Pub. No.: US 2022/0125553 A1
(43) Pub. Date: Apr. 28, 2022

(54) Methods for Direct Printing of Orthodontic and Dental Appliances onto the Teeth of a Patient

(71) Applicant: Marc Lemchen, New York, NY (US)

(72) Inventor: Marc Lemchen, New York, NY (US)

(21) Appl. No.: 17/489,129

(22) Filed: Sep. 29, 2021

Related U.S. Application Data
(60) Provisional application No. 63/106,252, filed on Oct. 27, 2020.

(51) Int. Cl. A61C 5/30; A61C 9/00; A61C 7/00; A61C 13/00; A61C 7/08; A61C 5/77; A61C 7/16; A61C 13/08; B33Y 50/00; B33Y 10/00; B33Y 80/00

(52) U.S. Cl. CPC A61C 5/30; A61C 9/0053; A61C 7/002; A61C 13/0019; A61C 7/08; B33Y 80/00; A61C 7/16; A61C 13/082; B33Y 50/00; B33Y 10/00; A61C 5/77

(57) ABSTRACT

A method for directly printing orthodontic and dental appliances on the teeth of a patient. The tooth is scanned with a scanner which is communicated with a computer controller configured to recognize the anatomy of the tooth. The surface of a tooth of the patient is then prepared in order to receive the type of appliance designated for the patient. Using image data from the print head, the computer controller prepares a three-dimensional blueprint or design of an appropriate orthodontic or dental appliance. The computer controller controls the type and amount of material that is distributed through the print head only when the print head passes over the recognized anatomy so as to directly print the corresponding appliance onto the tooth layer by layer as the print head makes successive passes over the surface of the tooth.

### RELATED APPLICATIONS

The present application is a non-provisional of U.S. provisional application Ser. No. 63/106,252, filed on Oct. 27, 2020 which is incorporated herein by reference in its entirety and to which priority is claimed pursuant to 35 USC 119.

### BACKGROUND

### Field of the Technology

The invention relates to methods and apparatus for use in orthodontics and dentistry, in particular three dimensional printing of orthodontic and dental appliances directly onto the teeth of a patient.

### Description of the Prior Art

Three-dimensional printing of parts and objects for many kinds of applications wherein the part or object was previously machined or molded is well known. For example, everything from picture frames, toys, and trays or racks are frequently printed on increasingly more efficient and cost-effective 3D printers. 3D printers however require a pre-existing or prepared file or blueprint in order print the 3D object. 3D printing has evolved quickly over the last few years, especially within the practice of dentistry and orthodontics. It is now possible to print in 3D biocompatible materials and construct devices which can safely be worn in the mouth. 3D printers use various methods including filament printing and inkjet printing which produce appliances or components that are matched to the color of the patient's teeth, including crowns and complete dentures.

More recently, handheld printers allowing users to quickly and efficiently print onto an object have been used in variety of industries including clothing design and shipping. A line of text or a graphical element is first entered or uploaded into the handheld printing device along with an ink or toner cartridge. The user can then quickly and repeatedly print the selected text or graphical element across the surface or material of nearly any object. The handheld printer is however entirely reliant on the skill and judgment of the user and does not provide a means for feeding direct input from the surface to be printed on

In the field of orthodontics and dentistry, in the past the standard current practice when applying an orthodontic or dental appliance is to first produce either a physical model or a three dimensional image of the patient's teeth using an intra-oral scanner. To obtain a physical model, an impression of the patient's teeth is first taken by conventional means, a plaster model of the teeth is then made from the impression, and then a dental and orthodontic appliance, crown or other dental element is handcrafted in a dental lab by a skilled dental lab technician. This process is time consuming, messy, and can highly unpleasant for the patient. Today, optical intra oral scanning and measurement of teeth in dental and orthodontic applications using a scanner is well-known and is quickly becoming the state of the art as its accuracy and ease of use increases. Intra oral scanning employs a handheld wand or probe which is inserted into the patient's mouth which then creates a scan or digital impression of the patient's teeth as the user moves the wand about the patient's mouth. The intra oral scanner is not only more comfortable than prior means, but it is also faster and more accurate than a completed physical mold. However, even when an intra oral scanner is used, a skilled dental lab technician must still prepare a handcrafted and unique dental or orthodontic appliance based on the digital impression, thus slowing down and increasing the overall costs of treating a patient while also unintentionally inherently impacting accuracy to some degree.

What is needed is a method whereby the precision and speed of three dimensional optical scanning is combined with the convenience and customizability of three dimensional printing for both extra-oral and intraoral applications in dentistry and orthodontics. The method should also be easy to use so that highly skilled lab technicians are not required.

### BRIEF SUMMARY

The provided method includes mapping the selected tooth, teeth and/or mouth of a patient. Next, the needed orthodontic or dental appliance is designed in a computer that is based on the previously generated dental map. Given that the printed appliance is to be “permanently” printed into place, any tooth surfaces are prepared as needed. Finally, the designed and selected appliance is printed onto the patient's teeth using a hand held or automated print head. In this manner, appliances such as veneers are printed on a patient's teeth in real time. Bonded lingual retainers may be printed directly onto a patient's teeth, even before any braces are removed. Even a replacement tooth or crown that is connected to an adjacent tooth may be printed, all in real time.

The current invention provides a method for printing an appliance directly onto a surface of at least one tooth of a patient, the method itself including scanning the surface of the at least one tooth with an intra-oral scanner, generating a 3D image of the surface of the at least one tooth within a computer communicated with the handheld print head device, and then applying a 3D representation of the appliance to the generated 3D image to create a 3D printing blueprint. The appliance is then printed directly on the surface of the at least one tooth using the handheld print head according to the 3D printing blueprint.

In one embodiment, the method also includes preparing the surface of the at least one tooth before directly printing the appliance to the surface of the at least one tooth using the handheld print head according to the 3D printing blueprint. Here, preparing the surface of the at least one tooth may include cleaning, etching, reducing the structure of the tooth via drilling, or sealing the surface of the at least one tooth.

In another embodiment, generating a 3D image of the surface of the at least one tooth within a computer communicated with the handheld print head device specifically includes recognizing an anatomy corresponding to the at least one tooth. In this embodiment, directly printing the appliance on the surface of the at least one tooth using the handheld print head according to the 3D printing blueprint occurs only when the anatomy corresponding to the at least one tooth is recognized.

In yet another embodiment, the method also includes continually monitoring the position of the handheld print head device relative to the surface of the at least one tooth. The print head device may use the scanned anatomy of the tooth and any adjacent teeth or their related structures in order to orientate itself, or alternatively, the print head may recognize temporary anchor devices (TADs) or other appliances to accurately identify its own position and angle relative to the surface of the at least one tooth or patient.

In a further embodiment, directly printing the appliance on the surface of the at least one tooth using the handheld print head according to the 3D printing blueprint specifically includes depositing a plurality of layers of dental material on or about the surface of the at least one tooth until the appliance has been formed. In this embodiment, directly printing the appliance on the surface of the at least one tooth using the handheld print head according to the 3D printing blueprint may also include performing a plurality of passes over the surface of the at least one tooth with the handheld print head device, each one of the plurality of passes depositing at least one of the plurality of layers of dental material on or about the surface of the at least one tooth until the appliance has been formed. Additionally, depositing a plurality of layers of dental material on or about the surface of the at least one tooth until the appliance has been formed may further include automatically matching a color, texture, or translucency of the surface of the at least one tooth and of a tooth that is adjacent to the surface of the at least one tooth.

In yet another embodiment, scanning the surface of the at least one tooth specifically includes scanning a portion of a dental arch of the patient and wherein generating the 3D image of the surface of the at least one tooth comprises the scanned portion of the dental arch of the patient.

In a further embodiment, directly printing the appliance on the surface of the at least one tooth using the handheld print head according to the 3D printing blueprint may specifically include directly printing a veneer, a crown, an aligner attachment, a retainer, or an orthodontic bracket on the surface of the at least one tooth.

The invention further provides a method for printing an appliance directly onto a surface of at least one tooth of a patient. The method itself includes scanning a first portion of the patient's mouth which is to receive the appliance, generating a map of the first portion the patient's mouth within a computer communicated with a portable print head, and then customizing the appliance in the computer based on the generated map. Next, a relative position is continuously determined between the first portion of the patient's mouth and a portable print head and then the customized appliance is printed onto the patient's teeth when the portable print head is determined to be within a predetermined range of the first portion of the patient's mouth.

In one particular embodiment, the method also includes preparing at least one tooth surface located within the first portion of the patient's mouth before the customized appliance is printed onto the patient's teeth.

In another embodiment, the method further includes matching a color, texture, or translucency of a first tooth surface located within the first portion of the patient's mouth to a second tooth surface.

In another specific embodiment, customizing the appliance in the computer based on the generated map includes dividing the appliance into a plurality of sequential layers, each layer being comprised of dental material. Here, the step of printing the customized appliance onto the patient's teeth when the portable print head is determined to be within a predetermined range of the first portion of the patient's mouth is done by repeatedly passing the print head within the predetermined range of the first portion of the patient's mouth and applying one of the plurality of layers of dental material onto the first portion of the patient's mouth each time the print head passes through the predetermined range of the first portion of the patient's mouth.

In an alternative embodiment, the method further includes scanning a second portion of the patient's mouth that is adjacent to the first portion of the patient's mouth which is to receive the appliance, generating a map of the second portion the patient's mouth within the computer communicated with the handheld print head device, and then printing the customized appliance onto the patient's teeth when the portable print head is determined to be within a predetermined range of both the first portion and the second portion of the patient's mouth, or alternatively, when the print head is determined to be within a predetermined range of a previously placed structure bonded or printed onto the teeth or placed into the patient's jaw bone such as a temporary anchor device (TAD).

In yet another embodiment, generating the map of the first portion the patient's mouth within a computer communicated with a portable print head specifically includes generating a map of a dental arch of the patient.

In a further embodiment, customizing the appliance in the computer based on the generated map also includes customizing a veneer, a crown, an aligner attachment, a retainer, or an orthodontic bracket based on the generated map.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is a flow chart demonstrating an embodiment of the current method for forming an appliance directly on the patient's teeth using a handheld print device.

FIG. 2 is a flow chart demonstrating how the current method maintains a selected portion of the patient's teeth in view so as to properly deposit of a layer of dental material each time the handheld print device is passed over the selected portion of the patient's teeth.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

The method of the current invention employs a dental printer which makes use of one or more three-dimensional printing technologies. Instead of building the object on a “build plate” within a table top printer however, the printing is performed with a print head that is passed multiple times over the surface of a patient's tooth or teeth.

Specifically, the current invention provides a method for directly printing dental and orthodontic appliances including but not limited to crowns, veneers, and aligner attachments onto the teeth of a patient. The method is performed with a print head which is hand held by a dentist or which may be controlled automatically by a robotic arm or automated system. The print head may itself in one embodiment comprise a conventional dental intra-oral scanner and is communicated with a computer controller and related software which is configured to recognize the anatomy of a tooth or a tooth which was been previously prepared to receive a retainer, or other orthodontic or dental appliance. In a related embodiment, the intra-oral scanner may instead be a separate component so that the operator first scans the patient with the intra-oral scanner before performing a separate series or sequence of passes with the handheld print head. The computer controller may be a traditional personal computer located within the user's office or it may be a network of computers or a cloud-based application distributed over a network such as the internet. The print head may be directly wired to the computer controller or is wirelessly connected through a WiFi or BlueTooth® connection. The print head comprises a plurality of video, LiDAR, or sonic sensors which the computer controller uses to not only to initially recognize the surface of the tooth, but also to continuously monitor the relative distance from the print head to the tooth surface located in three dimensions as well as to continuously determine whether or not that the print head is disposed over the selected area or tooth anatomy in order to accurately control the deposition of the resin or other dental material to produce a shape which adheres directly to the prepared, i.e. an etched or “carved”, surface of the tooth. The scanner within the print head may be a conventional scanner known in the art such as an iTero brand scanner as made by Align Technology Inc., San Jose, Calif.

While scanning, the print head is first passed over the teeth and the surrounding dental anatomy which is to have the dental appliance applied. After multiple passes have been completed by the scanner, a complete 3D image of the patient's tooth or teeth or portion of the patient's mouth is generated. The user then has the option of starting with a preparation of the tooth's surface by using the print head to apply a cleaning, etching, or sealing fluid onto the tooth in order to prepare the tooth for a subsequent crown, veneer, attachment for an aligner, or an orthodontic bracket. Specifically, the operator may first apply a means to clean and/or cut away dental material such as an air abrasion tool or a drill and then rinses the resulting debris away with a water spray or jet nozzle. Any remaining moisture or debris is then removed via a suction tube and then dried with a fan or other drying device. Next, an etching solution is applied, followed by another round of rinsing and then drying. A sealing solution is then applied, thereby finishing the preparation step and readying the surface of the tooth for the printing step where an acrylic or resin is applied over a series of layers. The air abrasion tool, drill, water nozzle, suction tube, fan, or any other dental or orthodontic tool used to prepare the surface of the tooth may be coupled to or incorporated into the print head device, or alternatively, may be kept as a separate component which may be used in conjunction or sequence with the print head device.

Once the tooth's surface has been prepared, the collected scanning data is used to define the object or appliance to be printed on the patient's teeth. Additionally, enough of the adjacent tooth or dental arch data is also scanned and analyzed so as to ensure that the object to be printed is accurately placed. The scanned data is transferred to the printing program controlling the three-dimensional printer. The attachment, veneer, crown, or retainer to be applied is designed from the collective scanned image using available software such as that produced by Exocad GmbH of Darmstadt, Germany; 3Shape NS of Copenhagen, Denmark; Mesh Maker brand software from Chetu of Plantation, Florida; AutoCad brand software from Autodesk Inc. of San Rafael, Calif.; or others.

The printing of the dental appliance is similar in concept and function to handheld fabric printers now in use, such as the EBS Handjet brand printer made by EBS Ink-Jet Systems Poland Sp. of Wroclaw, Poland, or Reiner Handheld Inkjet brand printer as distributed by Automated Marketing Inc. of Armonk, N.Y. While the print head continually monitors where it is relative to the anatomy of the tooth surface, the user actuates the print head to deposit multiple layers of dental material which make up or constitute the veneer, crown, bracket, or attachment. The computer controller uses the scanned image data received from the scanner to further automatically match the color, texture, and/or translucency of the surrounding surface of the tooth, an adjacent tooth, or another preselected or designated portion of the model by automatically controlling which available materials or colors of materials are emitted from the print head as it passed over the surface of the tooth, thereby essentially “painting” the veneer, crown, bracket or attachment directly onto a tooth.

In one specific embodiment, a curing light, either incorporated into the print head device or used as a separate component, is passed over the patient's tooth or teeth after each one of the plurality of layers of resin or acrylic is applied by the print head. For example, after depositing one of the layers used to form the designated appliance, the print head may be actuated so as to stop applying the resin or acrylic material and then activate a curing light. The print head is then passed over the surface of the tooth or teeth in an opposing direction so that the curing light may cure or harden the previously deposited resin/acrylic layer. Once a curing pass has been completed, the curing light is deactivated and the print head is once again passed back over the tooth or teeth with the print head applying the next or another resin/acrylic layer within the overall appliance printing process. Curing the surface of the tooth or teeth between each of the printed layers as opposed to a final curing step at the end of the printing process leads to a far more accurately formed appliance which is easier to correct and customize throughout the printing process.

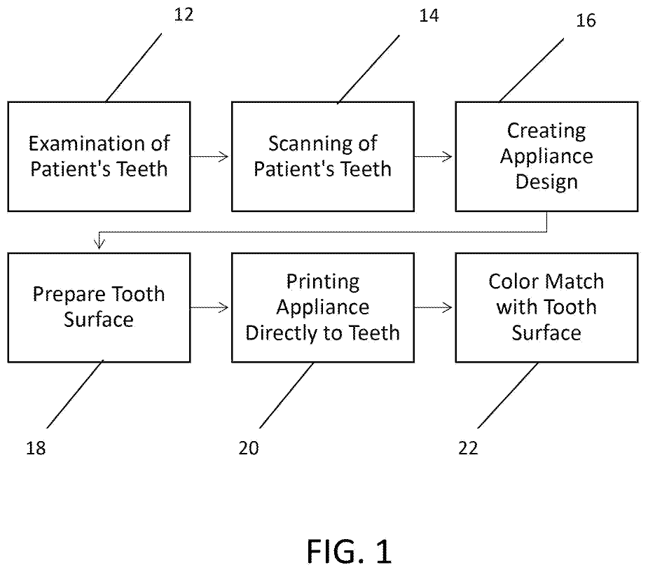

Turn now to the flow chart of FIG. 1 which outlines the current method 10 in specific detail while specifically applying or printing a plurality of attachments for an aligner treatment regimen directly onto a patient's teeth. While aligner attachments are currently disclosed, it is to be expressly understood that additional or alternative dental and orthodontic appliances such as veneers, crowns, orthodontic brackets, or other appliances now known or later devised which are applied directly to a patient's teeth are explicitly contemplated and may be incorporated into the current method without departing from the original spirit and scope of the invention. For example, it is expressly contemplated that the method of the current invention may be used to print or build any number of different dental or orthodontic appliances which are commonly known and which traditionally require fabrication by skilled technician in a lab. For example, the computer controller may contain software which allows veneers to be printed directly on the teeth of a patient in real time or print retainers directly on the patient's teeth even before any braces are removed. In another example, the current method may be employed to replace a missing tooth by printing in real time an entire replacement tooth which is connected or coupled to an adjacent tooth.

After a patient has been initially examined by a user and an aligner regimen or treatment has been determined in step 12, the user scans the tooth or teeth to be treated along with any surrounding teeth or adjacent tissue within the patient's mouth in step 14 using the intra-oral scanner which may be incorporated into the handheld print head or which may exist as a standalone component or device. A 3D image of the scanned tooth or teeth is then formed within a printing software program contained within the computer controller that is coupled or paired with the print head. This formed 3D image produces an image which corresponds to a first portion or target within the patient's mouth. The first portion or target serves as a type of guidepost or marker for the computer controller to constantly and consistently monitor for and recognize as the print head passes over the patient's dental anatomy. The first portion or target may contain or be based on anatomical features of the patient including but not limited to surfaces of the patient's teeth and gums, however in a further embodiment, other preexisting dental or orthodontic appliances including but not limited to braces or temporary anchor devices (TADs) may be included in order to signal to the computer controller that the selected or targeted area is in fact presently disposed directly beneath the print head device.

In step 16, a separate software program or software suite stored within the computer controller, such as that of Invisalign of San Jose, Calif., ULab Systems Inc. of San Mateo, California, SureSmile of Dallas, Tex., Clear Correct of Round Rock, Tex., or Star Aligners of Wichita Falls, Texas uses the scanned data to indicate specifically where the attachments are to be placed on the patient's teeth in order to interlock with the aligners in each instance. The scanned data comprising the 3D appliance to be printed is then returned to printing software program stored within the computer controller which then uses the scanned data comprising the 3D appliance to create a 3D printing blueprint which contains all the necessary instructions for the print head to print the attachments on the selected teeth in a sequential or progressive manner. Specifically, the printing software program divides or slices the 3D data defining the appliance to be printed as well as enough of the data defining the adjacent teeth or dental arch data so that the appliance may be printed at an appropriate and accurate location on the patient's teeth.

Next, in step 18, the user may prepare the surface of the tooth or teeth which the appliance is to be printed on. Where the printed appliance is a series of attachment points for aligners, the user may use the print head to clean the surface of the teeth via a cleaning solution or air abrasion tool dispensed by the print head. In addition to cleaning the surface of the tooth, the area where the appliance to be printed may be prepared by etching or sealing the surface as is required by the specific to be printed appliance as discussed above. The materials for etching or sealing the surface may also be dispensed through or via the print head.

Once the surface of the tooth has been properly prepared, the user in step 20 passes the print head multiple times over the patient's tooth or teeth while actuating the print head remains on or otherwise in a scanning mode. The printing software program stored within the computer controller accurately deposits sequential or sequenced layers of material or composite according to the created 3D printing blueprint. Specifically, with the assistance of continuous incoming real time image and sensor data, the computer controller selectively actuates the print head to deposit material when the print head is directly disposed over the selected or targeted area or anatomy which is to receive the appliance. In other words, each time the print head is passed over the first portion or target within the patient's mouth, the print head deposits a corresponding one of a plurality of layers of material or composite directly onto the prepared surface of the tooth only when it is determined that the print head is in fact disposed over the selected anatomy represented within the first portion or target. The computer controller continually adjusts as printing of the appliance progresses, namely with each successive layer building upon the previous one until the attachment has been fully formed as determined by the computer controller. If it is detected that the print head drifts outside of the selected area or anatomy, any application of the dental material immediately stops, thereby preventing dental material from being erroneously applied to a different tooth or portion of the patient's mouth.

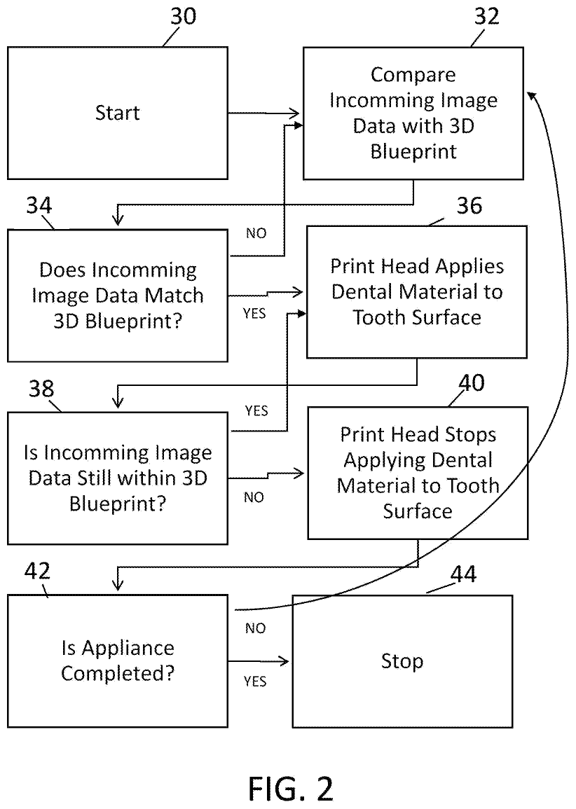

Turn now to a specific embodiment of how the print head device operates in FIG. 2. After sufficiently preparing the tooth surface as discussed above, the user first turns on the scanner and/or the print head device at step 30. Thereafter, an automatic tracking program in the computer controller to which the scanner and the print head device is communicated, constantly compares the real time incoming image data with the data associated with the created 3D printing blueprint in step 32. The incoming image data from the scanner may comprise anatomical features such as different surfaces of the patient's tooth or teeth, or it may include other distinguishing features such as other pre-existing dental or orthodontic appliances. Image tracking programs are well known to the art.

In step 34, the computer controller determines whether or not the incoming image data matches the previously formed 3D printing blueprint. If the incoming image data does not match the 3D printing blueprint, the computer controller returns to step 32 and continues to analyze the incoming image data. If the incoming image data does match the 3D printing blueprint, the print head device is activated and a predetermined amount of dental material such as acrylic or resin is emitted from the print head device in step 36 as the user moves the print head across the selected anatomy or tooth surface. Next, in step 38, the determination within the computer controller is made as to whether or not the incoming image data continues to match or sync up with the data within the 3D blueprint. If so, the computer controller returns to step 36 so as to continue emitting the dental material. However if the incoming image data and the 3D blueprint no longer match, the method proceeds to step 40 and the print head device is then instructed to deactivate and all emission of dental material stops. The computer controller then determines in step 42 if the selected appliance is completed or fully formed according to the created 3D blueprint. If the appliance is complete or finished, the print head is turned off in step 44. If the appliance is not complete however, the computer controller returns the print head and user back to step 32 so that the process may be repeated. It is in this fashion that each layer of the plurality of layers dictated by the 3D blueprint is sequentially deposited or “printed” of the surface of the tooth or teeth until the entire designed appliance has been formed or bonded to the patient's tooth or teeth.

Returning to FIG. 1, in another embodiment it should be noted that step 18 may be skipped completely if preparation of the tooth surface is not needed. Instead, the user may begin directly applying the appliance via the print head in step 20.

In one particular embodiment, the printing material which is dispensed from the print head is automatically color matched to the rest of the surface of the patient's tooth or adjacent teeth by the computer controller. Any color matching may occur at the same time as the attachments are deposited in step 20 or alternatively, the print head may deposit a color matching coat of material after the attachment has been fully formed in step 22. Additionally in step 22, the computer controller may be optionally configured to use the incoming image data from the intra-oral scanner to mimic the natural gradations in color and translucency of the patient's teeth or otherwise match the color associated with the image data received from the intra-oral scanner.

The current method as illustrated in FIGS. 1 and 2 eliminates the need to separately scan the patient's teeth and then wait for an appropriate or corresponding appliance to be made by a technician in a lab. For example, for any aligner or set of aligners, it is standard practice to often place attachments on the patient's teeth in order to enhance the “grip” of the aligner to the tooth, thereby improving the tracking accuracy of the tooth with sequential aligners. Currently, an attachment template is used to prepare the surfaces of the teeth to receive the attachments by filling the attachment template with composite, placing the attachment template on the patient's teeth, light curing the composite, and then removing the attachment template thereby leaving the attachments accurately placed on the tooth or teeth. Instead, in the current method, as the print head is passed over the tooth multiple times, each pass lays down a successive layer of dental composite in a specified position and with a specified shape as determined by the computer controller. It is in such a fashion that attachments may be quickly and accurately printed directly onto a patient's tooth or teeth without having to first produce a model of the patient's teeth, make a template based on the model, place a composite in the template, and then cure the template in order to produce the attachments which hopefully bond to the teeth when the template is removed. Instead, the method of the current invention provides more precise attachments and or appliances which are more likely to better adhere to the tooth.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

I claim:

1. A method for printing an appliance directly onto a surface of at least one tooth of a patient, the method comprising:
scanning the surface of the at least one tooth with an intra-oral scanner;
generating a 3D image of the surface of the at least one tooth within a computer communicated with the handheld print head device;
applying a 3D representation of the appliance to the generated 3D image to create a 3D printing blueprint; and
directly printing the appliance on the surface of the at least one tooth using the handheld print head according to the 3D printing blueprint.

2. The method of claim 1 further comprising preparing the surface of the at least one tooth before directly printing the appliance to the surface of the at least one tooth using the handheld print head according to the 3D printing blueprint.

3. The method of claim 2 wherein preparing the surface of the at least one tooth comprises cleaning, etching, reducing a structure of the at least one tooth, or sealing the surface of the at least one tooth.

4. The method of claim 1 wherein generating a 3D image of the surface of the at least one tooth within a computer communicated with the handheld print head device comprises recognizing an anatomy corresponding to the at least one tooth.

5. The method of claim 1 further comprising continually monitoring the position of the handheld print head device relative to the surface of the at least one tooth.

6. The method of claim 1 wherein directly printing the appliance on the surface of the at least one tooth using the handheld print head according to the 3D printing blueprint comprises depositing a plurality of layers of dental material on or about the surface of the at least one tooth until the appliance has been formed.

7. The method of claim 6 wherein directly printing the appliance on the surface of the at least one tooth using the handheld print head according to the 3D printing blueprint comprises performing a plurality of passes over the surface of the at least one tooth with the handheld print head device, each one of the plurality of passes depositing at least one of the plurality of layers of dental material on or about the surface of the at least one tooth until the appliance has been formed.

8. The method of claim 6 wherein depositing a plurality of layers of dental material on or about the surface of the at least one tooth until the appliance has been formed comprises automatically matching a color, texture, or translucency of the surface of the at least one tooth.

9. The method of claim 8 further comprising matching a color, texture, or translucency of a tooth adjacent to the surface of the at least one tooth.

10. The method of claim 4 wherein directly printing the appliance on the surface of the at least one tooth using the handheld print head according to the 3D printing blueprint only when the anatomy corresponding to the at least one tooth is recognized.

11. The method of claim 1 wherein scanning the surface of the at least one tooth comprises scanning a portion of a dental arch of the patient and wherein generating the 3D image of the surface of the at least one tooth comprises the scanned portion of the dental arch of the patient.

12. The method of claim 1 wherein directly printing the appliance on the surface of the at least one tooth using the handheld print head according to the 3D printing blueprint comprises directly printing a veneer, a crown, an aligner attachment, a retainer, or an orthodontic bracket on the surface of the at least one tooth.

13. A method for printing an appliance directly onto a surface of at, least one tooth of a patient, the method comprising:
scanning a first portion of the patient's mouth which is to receive the appliance;
generating a map of the first portion the patient's mouth within a computer communicated with a portable print head;
customizing the appliance in the computer based on the generated map;
continuously determining a relative position between the first portion of the patient's mouth and a portable print head; and
printing the customized appliance onto the patient's teeth when the portable print head is determined to be within a predetermined range of the first portion of the patient's mouth.

14. The method of claim 13 further comprising preparing at least one tooth surface located within the first portion of the patient's mouth before the customized appliance is printed onto the patient's teeth.

15. The method of claim 13 further comprising matching a color, texture, or translucency of a first tooth surface located within the first portion of the patient's mouth to a second tooth surface.

16. The method of claim 13 wherein customizing the appliance in the computer based on the generated map comprises dividing the appliance into a plurality of sequential layers, each layer being comprised of dental material.

17. The method of claim 16 wherein printing the customized appliance onto the patient's teeth when the portable print head is determined to be within a predetermined range of the first portion of the patient's mouth comprises:
repeatedly passing the print head within the predetermined range of the first portion of the patient's mouth; and
applying one of the plurality of layers of dental material onto the first portion of the patient's mouth each time the print head passes through the predetermined range of the first portion of the patient's mouth.

18. The method of claim 13 further comprising:
scanning a second portion of the patient's mouth that is adjacent to the first portion of the patient's mouth which is to receive the appliance;
generating a map of the second portion the patient's mouth within the computer communicated with the handheld print head device;
printing the customized appliance onto the patient's teeth when the portable print head is determined to be within a predetermined range of both the first portion and the second portion of the patient's mouth; or
printing the customized appliance onto the patient's teeth when the portable print head is determined to be within a predetermined range of a pre-existing appliance disposed on the patient's teeth.

19. The method of claim 13 wherein generating the map of the first portion the patient's mouth within a computer communicated with a portable print head comprises generating a map of a dental arch of the patient.

20. The method of claim 13 wherein customizing the appliance in the computer based on the generated map comprises customizing a veneer, a crown, an aligner attachment, a retainer, or an orthodontic bracket based on the generated map.
